(19) United States
(12) Patent Application Publication
(10) Pub. No.: US 2023/0013357 A1
(43) Pub. Date: Jan. 19, 2023

(54) EXAMINATION DEVICE AND EYE EXAMINATION METHOD

(71) Applicant: MEDIZINISCHE UNIVERSITÄT WIEN, Wien (AT)

(72) Inventors: Christoph MITSCH, Wein (AT); Hannes KAUFMANN, Wein (AT); Emanuel VONACH, Wein (AT); Sabine KOINIG, Wein (AT); Andreas REITNER, Wein (AT); Berthold PEMP, Wein (AT)

(73) Assignee: MEDIZINISCHE UNIVERSITÄT WIEN, Wien, AUSTRIA

(21) Appl. No.: 17/786,522

(22) PCT Filed: Dec. 1, 2020
(86) PCT No.: PCT/EP2020/084018

§ 371 Date: Jun. 16, 2022

(30) Foreign Application Priority Data
Dec. 17, 2019 (EP) 19216778.1

(51) Int. Cl. A61B 3/11; A61B 3/113; A61B 3/14; A61B 3/00; A61B 5/11; A61B 5/00

(52) U.S. Cl. CPC A61B 3/112; A61B 3/113; A61B 3/14; A61B 3/0091; A61B 5/1114; A61B 3/0033; A61B 3/0025; A61B 5/6803; A61B 2560/0431

(57) ABSTRACT

The invention relates to an examination device (1), a method for an automated examination of at least one eye (4, 4′) of a person, a computer program product and the various uses of the examination device (1).

The invention relates to an examination device and a method for eye examination.

Testing the eye movements, visual field, color vision, and pupillary response are fundamental examinations for making accurate medical diagnoses. The data obtained during these examinations support the correct diagnosis and, based on it, the therapeutic management of many ophthalmological, neurological and ear, nose and throat disorders.

Currently, eye movement disorders and squint angle measurements in eye departments and doctor's offices are mainly tested by specially trained medical staff, such as orthoptists in particular. Accuracy and reproducibility strongly depend on the experience of these personnel. Particularly subtle changes, such as rotary nystagmus in specific viewing directions, can easily be overlooked and sometimes quantitative assessment is inaccurate and subject to error.

Color perception testing is examined with the saturated or desaturated panel D-15 test. However, a disadvantage of this test is that only rough summative quantitative result indices could be derived from it and furthermore the resolution or screening (15 color plates from the entire color spectrum are to be arranged in the correct order) of the result is often too rough to be able to use it as a sensitive progression parameter for disorders affecting the sense of color.

The disturbed color saturation, which is characteristic for many diseases of the optic nerve, can therefore currently not be quantified exactly.

Pupillography is currently performed using special cameras and computer systems. Both illumination in terms of reflex stimulation and testing of illumination conditions are the responsibility of the examiner and are therefore difficult to standardize and quantify.

Visual field examinations are often performed at previously mentioned institutes and have to be mastered by the persons in a sitting position until now. For persons with limited mobility with special, especially neurological issues, this examination with conventional means is currently only roughly possible with confrontation perimetry. Confrontation perimetry, or finger perimetry, is limited to testing the outer limits of the visual field and does not provide information about defect depths and does not allow reliable identification or exclusion of isolated scotomas.

For example, methods of perimetry are known in which the visual field of one eye is systematically measured with a computer perimetry device, such as the Zeiss Humphrey Field Analyzer, while the other eye is covered. For this purpose, the person looks into a hemisphere of the computer perimetry device with the eye to be measured and focuses a stationary light spot. Subsequently, measuring light spots at different positions and with different brightness are displayed in the hemisphere by computer control. The person confirms each recognized measuring light spot by pressing a button on an input device.

However, the disadvantage of such methods is that the person's eye to be measured must always be focused precisely on the stationary light spot for the measurement to be correct. Furthermore, due to the design of conventional computer perimetry devices, persons must undergo the measurement while seated. Therefore, measuring the visual field of elderly or ill persons with limited mobility is very difficult with conventional computer perimetry devices, if possible at all, and is subject to great inaccuracy. Furthermore, measurement accuracy requires that the computer perimetry devices be used under controlled lighting conditions. Also, these expensive, bulky and heavy devices are not at all designed for portable use.

Furthermore, methods are known in which eye movement, strabismus or color vision can be checked. With conventional methods, however, the result of these examinations always potentially exhibits a high degree of inaccuracy, since these examinations are also dependent on the examiner and the examination environment, among other things.

The object of the invention is among others to overcome these and other disadvantages of the prior art. In particular, it is the object of the invention to provide an examination device and a method with which the eyes of a person can be examined easily, quickly and accurately. Another object of the invention is to provide a device and a method with which the various examinations can be carried out in any position of the person and independent of location and environment.

These and other objects are solved by a device and a method having the features of the independent patent claims.

The invention relates to an examination device for examining at least one eye of a person, wherein the examination device comprises at least one optical sensing device, in particular a camera or an infrared sensor, which is configured to sense eye movements and preferably eye positions of at least one eye of a person.

The examination device comprises at least one optical display device, in particular a screen or a projection device, which is adapted to display or project images, and is adapted to be connected to an electronic data processing unit for storing and/or for automated evaluation of the eye movements and/or eye positions in relation to the displayed images, or comprises such an electronic data processing unit.

According to the invention, the examination device is adapted to display an image by the display device prior to sensing the eye movements and eye positions by the sensing device or prior to displaying an image by the display device in order to bring at least one eye into a predefined eye position.

The optical sensing device may be designed as an infrared camera or infrared sensor, which is configured to sense eye movements, eye positionings, eye positions and pupils of the person's eyes with invisible infrared light (so-called eye tracker).

The optical display device may be designed as a screen, for example an LCD screen, for displaying images. However, the display device may also be designed as a projection device, in particular a laser, which projects the information onto the retina of the eye. It may be provided that the projection device projects the image directly into the eye of the person or projects the image via a mirror surface, the mirror surface being arranged in front of the eye of the person.

The display device may comprise one screen having a resolution of 2560 pixels by 1440 pixels, or two screens each having a resolution of 1280 pixels by 1440 pixels. The resolution of the display device may be in the range of 160 pixels by 120 pixels up to and including 15360 pixels by 8640 pixels, in particular in the range of 640 pixels by 480 pixels up to and including 4096 pixels by 2160 pixels. The frame rate of the display device may be in the range of 40 fps up to and including 500 fps, in particular in the range of 50 fps up to and including 150 fps.

The horizontal field of view of the display device may be in the range of 50° up to and including 200°, in particular in the range of 90° up to and including 200°.

The vertical field of view of the display device may be in the range of 50° up to and including 130°, in particular in the range of 90° up to and including 130°. In other words, the display device may cover substantially the entire field of view of the person.

Optical lenses may be arranged between the eye of the person and the at least one display device, the at least one screen of the display device and/or the images displayed by the display device, in particular directly, in front of the eye. The optical lenses may be configured to allow the person's eye to focus on the displayed images. In other words, it is optionally provided that the lenses are arranged such that the eyes of the person look through the lenses at the at least one display device, at the at least one screen of the display device, and/or at the images displayed by the display device. In particular, the at least one optical lens may be designed as a spherical lens, an aspherical lens, or a Fresnel lens.

The examination device, in particular the display device, may also comprise two separate sensing devices.

The sensing accuracy of the sensing device may be less than 5°, in particular less than 2°, preferably less than 1°. The sensing speed of the sensing device may be in the range of 50 fps up to and including 500 fps, in particular in the range of 90 fps up to and including 300 fps, and in particular 120 fps. The examination device may be portable and may be carried in a conventional carrying case. The weight of the head-mounted portion of the examination device and in particular the display device may be in the range of 10 g up to and including 2000 g, in particular in the range of 50 g up to and including 1000 g, preferably in the range of 100 g up to and including 750 g.

The examination device may be designed for connection to an electronic data processing unit for storage and automated evaluation of the eye movements, the eye positions, the interpupillary distance and/or at least one pupil size in relation to the displayed images. However, the examination device may comprise such an electronic data processing unit itself.

The data processing unit may be integrated into the examination device, or it may be a computer, smartphone or the like. In particular, the recorded, stored and/or evaluated data may be transmitted directly to a computer, smartphone or the like, in particular wirelessly.

The examination device may be configured to present various stimuli to the person to be examined. The eye movements, such as in particular the ductions, versions, and vergences, and their perturbations can be displayed or stored on time-position graphs or stimulus-viewing direction graphs for each eye. Furthermore, it is optionally possible to store and/or evaluate the movement responses of the eyes to nystagmus-inducing projected patterns, head posture, and/or head movement.

With the examination device, the currently most frequently used oculomotor, perimetric and pupillographic tests, as well as more specialized examinations of color vision and eye muscle function, can be performed quickly, easily, independently of location and in a standardized manner.

Due to the design of the examination device, these tests can also be performed on lying persons.

Furthermore, the examination device allows an examination to be performed independently of other environmental conditions, such as the brightness in the examination room.

Optionally, it is possible to design the examination device in such a way that the examination can be carried out quickly, accurately and independently of location in any position of the person, for example lying down, standing up or sitting down.

Optionally, it is provided that the examination device comprises at least one input device, such as a wireless button, which is configured to detect manual inputs of the person in relation to the displayed images, wherein the data processing unit is adapted to store and evaluate the sensed inputs in relation to the displayed images.

Optionally, it is provided that the examination device comprises a detection device, in particular a gyrometer, an acceleration sensor and/or a camera, preferably an outside-in and/or an inside-out camera system, which is configured to detect head movements of the person, the data processing unit being adapted to take into account the sensed head movements in the evaluation of the eye movements, the eye positions and/or the inputs.

Optionally, the examination device is or can be connected to a detection device, in particular a camera configured to detect the head movement.

When using an inside-out camera system, at least one camera configured to sense the position of the examination device in space and in particular the head movement may be arranged on the examination device itself. In the case of an examination device, preferably a plurality of cameras of this type, in particular between one and twenty, particularly preferably between two and ten, are arranged on the examination device.

When using an outside-in camera system, the examination device itself optionally does not have any cameras configured to sense the position of the examination device in the room and in particular the head movement.

When using such an outside-in camera system, the at least one camera configured to sense the position of the examination device in the room may be arranged at a distance from the examination device. In the case of outside-in camera systems, preferably a plurality of cameras of this type, in particular between one and twenty, particularly preferably between two and ten, are arranged at a distance from the examination device. Furthermore, the examination device may have at least one position flag, in particular at least one infrared LED, which can be detected by the cameras.

The data recorded by at least one camera may be transmitted to the data processing unit.

Optionally, it is provided that the sensing device or a separate optical sensor is configured to sense the pupil size of the eye of the person, the data processing unit being adapted to take into account the sensed pupil size in the evaluation of the eye movements, the eye positions and/or the inputs, and/or that the sensing device or a separate optical sensor is configured to sense the interpupillary distance, the data processing unit being adapted to take into account the sensed interpupillary distance in the evaluation of the eye movements, the eye positions and/or the inputs.

The sensing device or a separate optical sensor may be adapted to continuously and/or simultaneously measure the pupil of at least one eye and its diameter. It may be an infrared camera. This allows testing of the person's pupillary responses in a controlled, optically isolated, and therefore standardized environment.

Optionally, it is provided that the examination device comprises at least two spatially separated display devices, wherein the images displayed on one display device are only visible for one eye of the person and the images displayed on the other display device are only visible for the other eye of the person.

The examination device may comprise at least two spatially separated display areas, wherein a first display area is only visible for one eye of the person and a second display area is only visible for the other eye of the person. The examination device may be configured in such a way that one eye can see the image independently of the other eye, i.e., that a displayed image can only be seen by one eye and another displayed image can only be seen by the other eye. For this purpose, the respective visual range of each eye may be completely covered by the associated display device.

Optionally, the examination device may exclusively comprise one display device, in particular a screen extending over the visual range of both eyes, wherein one part of the display device is exclusively visible to one eye and another part of the display device is exclusively visible to the other eye.

Optionally, the examination device comprises exclusively one display area, the image displayed on that display area being visible only for the first eye of the person. In this case, it may be provided to cover the second eye such that light stimuli to the second eye are substantially prevented.

Further, the examination device may be configured such that only the images displayed by the examination device are visible for the eyes. This allows the examination device to perform an examination of the eyes in a fully controlled, optically isolated environment.

Optionally, it is provided that the examination device is adapted to be arranged on the head of a person and is or comprises in particular a portable visual output device and preferably is a head-mounted display, the head-mounted display comprising in particular two separately controllable display devices.

Preferably, the examination device covers the entire field of view of the person. Thus, the person can be isolated from other visual impressions of the environment while wearing the examination device. As a result, high measurement accuracy can be achieved under standardized examination conditions, even if the person moves during the examination. Furthermore, the method can be performed independently of external influences.

In particular, the examination device may be designed as a head mounted display, such as a so-called FOVE 0 with two spatially separated and separately controllable display devices and separate eye trackers in each case, as an HTC Vive Pro Eye, as a Google Glass or as an Intel Vaunt.

The invention further relates to a method for an automated eye examination using an examination device according to the invention, the method comprising the following steps: displaying an image by the display device and sensing an eye movement and/or an eye position of the eye by the sensing device; storing and/or evaluating the at least one sensed eye movement and/or eye position and the at least one displayed image by an electronic data processing unit connected to the examination device, in particular by a computer.

It may be provided that displaying an image by the display device and sensing an eye movement and/or an eye position by the sensing device are done substantially simultaneously. By sensing the eye positions, also the viewing direction of the eyes may be sensed.

It may be provided that displaying an image takes place before sensing an eye movement and/or an eye position; it may also be provided that displaying an image takes place after sensing an eye movement and/or an eye position.

It may be provided that the method is performed in an automated manner on a computer connected to the examination device, in particular wirelessly, or integrated into the examination device. Preferably, it may be provided that the method according to the invention is performed in an automated manner, in particular controlled by a computer. The data processing unit may be integrated into the examination device, in particular into the at least one display device.

The method steps may optionally be carried out once, twice or even several times during an examination.

With this method it may be possible to objectively, quantitatively and ultimately without the mandatory requirement of the presence of ophthalmologists or orthoptists, test eye movements and their disturbances, including possible torsional deviations and physiological and pathological nystagmus, the visual field of the person, the color sense and the pupillary responses of the person. With this method, conventional psychophysical examinations, which previously depended on feedback from the person being examined, can now be performed without feedback and, in particular, completely automatically.

Optionally, it is provided that the method according to the invention comprises the following steps: sensing an eye movement and/or an eye position of the eye by the sensing device, displaying an image in dependence on the sensed eye movement and/or the sensed eye position of the eye by the display device, storing and/or evaluating the at least one sensed eye movement and/or eye position and the at least one displayed image by an electronic data processing unit connected to the examination device, in particular by a computer.

Optionally, it is provided that the method according to the invention comprises the following steps:

displaying an image by the display device, sensing an eye movement and/or an eye position of the eye by the sensing device in dependence on the displayed image, storing and/or evaluating the at least one sensed eye movement and/or eye position in relation to the at least one displayed image by an electronic data processing unit connected to the examination device, in particular by a computer. The method steps may be carried out once, never or even several times during the examination of a person.

According to the invention, it is provided that before an eye movement and/or an eye position of the eye is sensed by the sensing device, an image is displayed by the display device in order to bring at least one eye into a predefined eye position, or that before an image is displayed by the display device, an image is displayed by the display device in order to bring at least one eye into a predefined eye position.

In particular, it may be provided that the displayed images comprise a first image component, in particular a first picture on which the eye is to focus, and/or a second image component, in particular a second picture. Preferably, it may be provided that the displayed images comprise the same first image component and a different second image component or no second image component, respectively. The first image component may be formed as a light spot, in particular as a focus point. The second image component may be formed as a light spot, in particular as a measuring point. The image components, in particular the light spots, may differ in their size, intensity, such as brightness, and/or display duration. Preferably, all image components are designed as light spots.

By displaying a first image with only one image component, in particular with only one light spot, it may be possible to bring the eye position of at least one eye into a predefined eye position. In particular, it is thereby possible to direct the viewing direction of at least one eye into a predefined area of the display device.

Optionally, it is provided that the viewing direction of the eye is first directed towards an edge region of the image area of the display device or towards an edge region of the display area.

Subsequently, a further image with an image component, in particular with a light spot, may be displayed, wherein the image component of the further image is spaced apart from the image component of the preceding, in particular the first, image. By directing the viewing direction, it may be possible to exploit the size of the image area of the display device or the size of the display area.

Optionally, it is provided that the method according to the invention comprises the following further steps: recording manual inputs from the person in relation to the displayed images by an input device connected to the examination device, for example a wireless pushbutton, storing and evaluating the sensed inputs in relation to the displayed images by the data processing unit.

The input device may be, in particular, a wireless pushbutton or a hand-held remote control configured to sense manual inputs from the person.

Optionally, it is provided that the method according to the invention comprises the following further steps: recording head movements of the person by a detection device connected to the examination device, in particular a gyrometer, an acceleration sensor and/or a camera, taking into account the sensed head movements by the data processing unit in the evaluation of the eye movements, eye positions and/or inputs. This may allow to prevent and/or take into account a distortion of the results caused by head movements of the person.

Optionally, it is provided that the examination device is arranged on the head of the person so that the examination device and the display device follow the head movements of the person.

Optionally, it is provided that the eye movements and the eye positions of both eyes of the person are sensed substantially simultaneously with one or more sensing devices, and/or that images are displayed substantially simultaneously for both eyes of the person.

Optionally, it is provided that the pupil size of the eye is determined using the sensing device or a separate optical sensor, wherein the eye movements, the eye positions, the sensed pupil size and/or the inputs are taken into account by the data processing unit during evaluation, and/or that the interpupillary distance is determined using the sensing device or a separate optical sensor, wherein the eye movements, the eye positions, the sensed interpupillary distance and/or the inputs are taken into account by the data processing unit during evaluation.

The eye movements, the eye positions of both eyes and thus the positioning of the eyes of the person relative to each other may be sensed substantially simultaneously using a single sensing device.

Likewise, it may be provided that the eye positions of both eyes of the person are sensed substantially simultaneously using multiple, preferably two, spatially separated sensing devices.

Optionally, images for both eyes of the person may be displayed substantially simultaneously. This allows simultaneous examination of both eyes, a so-called binocular examination. Furthermore, the distance between the pupils, the interpupillary distance, can be taken into account in the calculation and presentation of the results. According to the invention, the pupil sizes of the eyes can further be determined with the sensing device or with a separate optical sensor, wherein the eye movements, the eye positions, the sensed pupil size and/or the inputs may be taken into account by the data processing unit in the evaluation. In particular, this can reduce and/or prevent a distortion of the results.

Optionally, it is provided that the sensed eye movement of the person is also taken into account in the evaluation of the results. In particular, this can reduce and/or prevent a distortion of the results.

The invention further relates to a computer program product, in particular a computer-readable storage medium, on which a computer program for carrying out the method according to the invention and/or the use of the examination device according to the invention on a computer is stored.

The invention further relates to various uses of the examination device according to the invention for functional, in particular automated, eye diagnostics.

In the use of the examination device according to the invention for testing the duction, in particular for testing the eye movement, of a person, a first light spot may be displayed, in particular exclusively, on the at least one display device during a first measurement, wherein the eye position of at least one eye is sensed with the at least one sensing device when the eye focuses on the first light spot.

In a further measurement, another light spot may be displayed on the at least one display device, in particular exclusively, which is closer to an edge of the respective display device compared to the first light spot, wherein the eye position of the at least one eye is sensed with the at least one sensing device when the eye focuses on the other light spot.

Optionally, the head movement, pupil size and/or eye movement are sensed during and/or between the first and further measurements.

The sensed eye positions, head movements, pupil sizes and/or eye movements may be stored and/or evaluated by the data processing unit. Further, the eye positions of both eyes may be continuously output and/or stored by the data processing unit.

The sensed eye positions, head movements, pupil sizes and/or eye movements may be stored and/or evaluated by the data processing unit, wherein, if substantially no difference is detected between the sensed eye positions, a maximum eye movement may be output and/or stored by the data processing unit.

By sensing the eye movement, in particular the eye position, the maximum eye movement may be measured directly with the examination device. Thus, feedback from the person being examined, as in conventional examination methods, is not necessary.

By using the examination device to test the eye movement, static and kinetic fixation targets and other stimulus patterns may be presented to the person and the eye movement may be measured and/or recorded in a fully controlled, in particular closed and/or optically isolated, environment.

When using an examination device according to the invention for the perimetry of a person, a first eye position of the eye may be sensed during a first measurement by the at least one sensing device, wherein a first light spot is displayed on the display device in dependence on the sensed first eye position. In a further measurement, a second eye position of the eye may be sensed by the at least one sensing device, wherein a different light spot is displayed on the display device in dependence on the sensed second eye position.

Optionally, the first eye position is substantially identical to the second eye position so that in this case only the displayed images, in particular the light spots, are different.

Manual inputs of the person may be sensed in relation to the displayed light spots by the input device, whereby a head movement, an eye movement and/or the pupil size are optionally detected during and/or between the first and the further measurements.

The manual inputs of the person in relation to the displayed light spots may be stored and/or evaluated by the data processing unit taking into account the eye movement, the eye position, the head movement and/or the pupil size.

By this use, it becomes possible to perform perimetry of one or both eyes regardless of location and without guiding the eyes. In other words, the visual field of the eyes may be determined without the person having to concentrate on a fixed point while seated. Even on the contrary, it may be possible for the person to perform eye movements during the measurement, since the measuring points are displayed with respect to the respective present eye positioning, eye position and/or visual axis.

A measuring point may be displayed for the duration of 2 ms up to and including 5000 ms, in particular for the duration of 150 ms up to and including 1000 ms. Thus, the eye position may be assumed to be constant during the display of a measuring point.

By this use, visual field perturbations may be examined with a single-point stimulus program and a multiple-point stimulus program. Here, stimuli of configurable size, intensity and number may be projected or displayed at predetermined positions in the visual field of both eyes of examined persons.

The responses of the examined person may be recorded using an input device, in particular a wired hand-held remote control, and may be evaluated together with information about the position, size, duration and intensity (brightness, dB) of the triggering stimulus.

By using the examination device for the perimetry of a person, both eyes can be examined simultaneously without the environmental conditions having to be taken into account.

Optionally, the method according to the invention comprises the following steps:

In a first step, sensing a first eye movement and/or a first eye position of the eye by the sensing device.

In a further step, displaying a first image in dependence on the first eye movement and/or the first eye position by the display device.

In a further step, sensing a second eye movement and/or a second eye position of the eye by the sensing device.

In a further step, displaying a second image in dependence on the second eye movement and/or the second eye position by the display device.

In a further step, storing and evaluating the sensed eye movements and/or eye positions in relation to the displayed images by an electronic data processing unit connected to or incorporated into the examination device, in particular by a computer.

The examination device may also be used for saccade controlled and/or viewing direction flexible perimetry:

With the detection device, in particular an eye tracker, eye movements in response to images displayed by the display device, so-called stimuli, can be recorded and evaluated in relation to the position of the stimulus. In other words, for the purposes of the invention, a stimulus can be understood as an image displayed by the display device.

A saccade in the direction of the stimulus can be counted after a defined time, in particular within a predefined period of time, namely a so-called tolerance, as a positive reaction, namely that the patient has perceived the stimulus.

Subsequently, the next stimulus can be displayed in relation to the then current eye position, in particular the viewing direction of the at least one eye.

If necessary, one or more auxiliary stimuli may be displayed to direct the eye to an eye position and/or to change the viewing direction of the at least one eye from which further stimuli to be tested may be displayed.

When using an examination device according to the invention for deviation, in particular for strabismus diagnosis, of a person, a first image can be displayed to at least one eye, in particular to both eyes, of the person by the display device, the image being configured such that it appears to the person as one image.

Thereby, at least one image can be displayed to a person, in particular to at least one eye of the person, in particular both eyes of the person, at different angles to an eye axis, in particular at least one image in all cardinal directions.

This in particular automatic reciprocal cover test may be used to determine specific strabismus characteristics with special evaluations and novel display forms.

The eye movements and in particular the eye positions of both eyes may be sensed using the sensing devices, wherein optionally the eye movements, the head movements and/or the pupil size are detected during the measurement.

The data processing unit may calculate the difference of the sensed eye positions, and may take into account the eye movements, the head movements, and/or the pupil sizes of the eyes in the calculation.

By using the examination device in such a way, it may be possible to examine the movement, in particular the eye position, of both eyes simultaneously with a closed, in particular optically isolated, system. In other words, it may be possible to quantify pathological changes of the eye mobility and/or the eye positions relative to each other by displaying light spots at different positions.

By using the examination device for determining the positions of the eyes relative to each other, the eyes of the person may be alternately “covered”, in particular the fixation/fusion stimulus may be concealed. Meanwhile, however, the performed movements of both eyes may optionally be measured simultaneously.

When using an examination device according to the invention for color diagnosis of a person, an image with a substantially identical or slightly different color may be displayed preferably simultaneously on the display device visible for one eye and on the display device visible for the other eye.

The input device allows the saturation and brightness of the color of the images displayed on the display devices to be adjusted separately and manually by the person until the person subjectively perceives both images in an identical color. Optionally, the eye movements, the eye positions, the head movements and/or the pupil sizes may be detected during the measurement.

The data processing unit may calculate the difference in the saturation and the brightness of the images and may optionally take into account the eye movements, the eye positions, the head movements and/or the pupil sizes in the calculation.

This use permits to quantify the color perception of the person without external influences using a closed, in particular optically isolated, system. Optionally, the measuring accuracy is much higher than with conventional methods due to the fact that the person can only see one screen with one eye and the other screen with the other eye during the measurement.

This makes it possible to quantitatively test a person's sense of color or color perception by letting the person change the brightness and saturation of a color surface projected in the visual field of one eye until the subjective color perception appears the same in both eyes.

Optionally, it is provided that the examiner changes the brightness and/or saturation of the color seen by one eye of the person. In this case, the person may focus solely on the reproduction of the color.

When using an examination device according to the invention for testing a person's vergence, a first image may be displayed for both eyes of the person, wherein a first pupil size and/or a first interpupillary distance of the eyes is sensed using the sensing devices.

Then, a further image may be displayed for both eyes of the person, the picture being designed to appear closer or further away to the person than the first image, wherein a second pupil size and/or a second interpupillary distance of the eyes may be sensed using the sensing devices.

During the measurement, the eye movements, the eye position and/or the head movements may optionally be detected, and the difference of the sensed pupil sizes and/or the sensed interpupillary distances may be calculated by the data processing unit.

The calculation may take into account the eye movements, the eye position, and/or the head movements of the eyes.

When using an examination device according to the invention for pupillography of a person, a first image may be displayed for at least one eye, in particular both eyes, of the person, wherein the first image may optionally be black or white, and a first pupil size and/or a first interpupillary distance of the eyes may be sensed using the sensing devices.

Then, a further image may be displayed for at least one eye, in particular both eyes, of the person, the picture being designed to appear brighter or darker to the person than the first image, and a second pupil size and/or a second interpupillary distance of the eyes may be sensed using the sensing devices.

During the measurement, the eye movements, the eye position, the pupil size, the interpupillary distance and/or the head movements may optionally be detected. The data processing unit may calculate the difference of the sensed pupil size and/or the sensed interpupillary distance and may optionally take into account the eye movements, the eye position and/or the head movements of the eyes in the calculation. In particular, it is provided that the speed of the change of the pupil size of at least one eye is calculated, taken into account in the evaluation and/or output.

When using an examination device according to the invention for a person's flicker fusion, at least one eye, in particular both eyes, may be presented multiple times with a stroboscopic flashing image, in particular an object.

The blinking frequency of the image may be increased further, in particular initially, and preferably the blinking frequency is increased further and further so that the person can no longer perceive the blinking of the image as such. When the first threshold value is reached, i.e., the frequency above which the person can no longer perceive the blinking of the image as such, the person can make an input via the input device of the examination device.

The blinking frequency of the image may be slowed down, in particular subsequently, preferably the blinking frequency of the image is slowed down in such a way that the subject can perceive the blinking of the image as such again. When the second threshold value is reached, i.e., the frequency above which the person can perceive the blinking of the image as such again, the person can make an input via the input device of the examination device.

The responses of the examined person may be recorded using an input device, in particular a wired hand-held remote control, and may be evaluated together with information about the blinking frequency. These method steps may be carried out a few times in succession.

By these steps, the flicker fusion frequency of the person can be determined. It can be altered, in particular slowed down, just as in the case of a wide variety of visual pathway lesions.

In all of the previously described uses of the examination device, the movements of the eyes, the movements of the head, the interpupillary distance and/or the pupil sizes of the eyes may be taken into account. The eye and head movements, the eye position, the interpupillary distance and/or the pupil sizes may be output graphically in the form of time-position diagrams and time-size diagrams, respectively.

Further features according to the invention are optionally derived from the claims, the description of the exemplary embodiments and the figures. The invention is now further explained in more detail on the basis of exemplary and non-limiting exemplary embodiments.

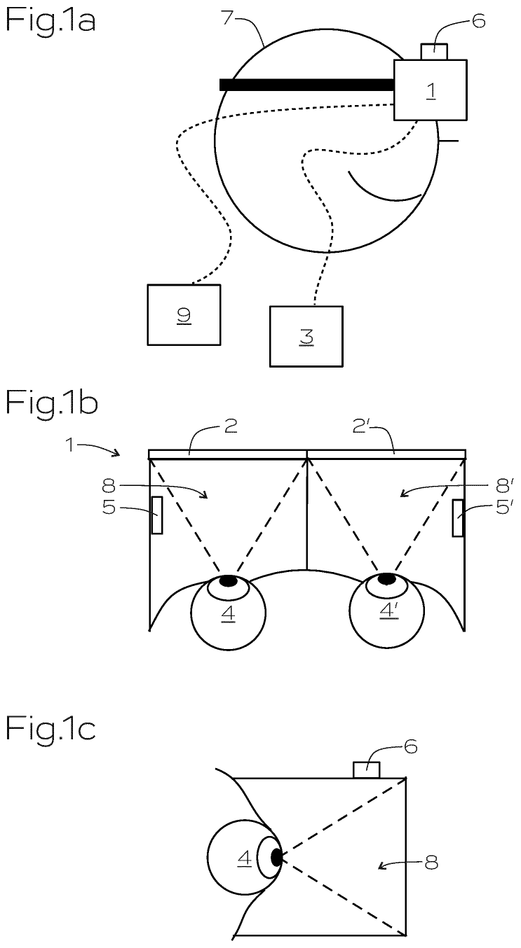

FIGS. 1a, 1b and 1c show a schematic graphic view of a first embodiment of the examination device according to the invention in different views, which is configured to carry out the method according to the invention;

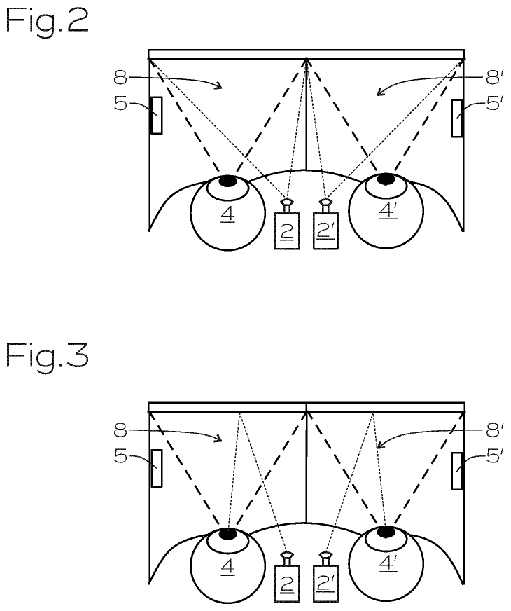

FIG. 2 shows a schematic graphic view of a second embodiment of the examination device according to the invention, which is configured to carry out the method according to the invention;

FIG. 3 shows a schematic graphic view of a third embodiment of the examination device according to the invention, which is configured to carry out the method according to the invention;

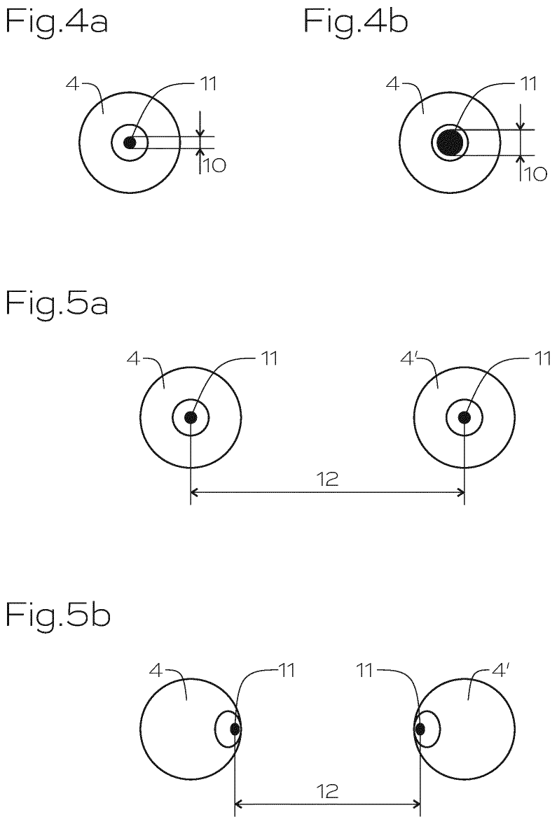

FIGS. 4a and 4b show schematic diagrams of a person's eye and the respective pupil size;

FIGS. 5a and 5b show schematic diagrams of the interpupillary distance of a person's eyes;

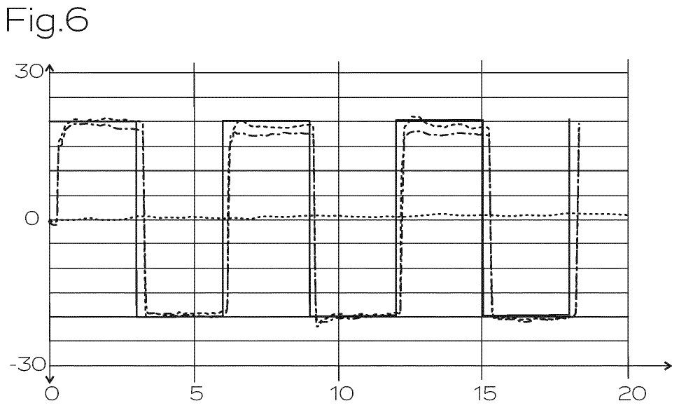

FIG. 6 shows a schematic view of a result of a test of the saccades with the examination device according to the invention;

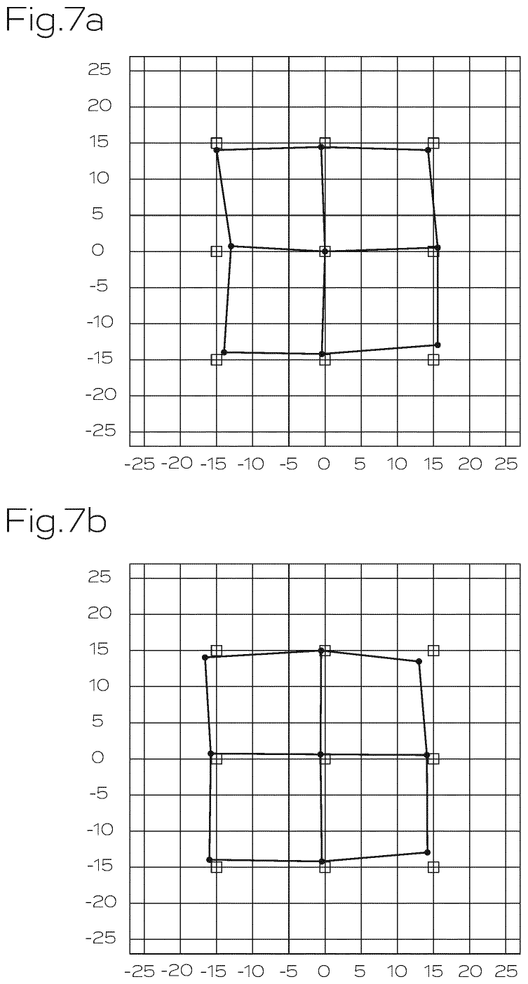

FIGS. 7a and 7b show schematic diagrams of a result of a strabismus diagnosis with the examination device according to the invention;

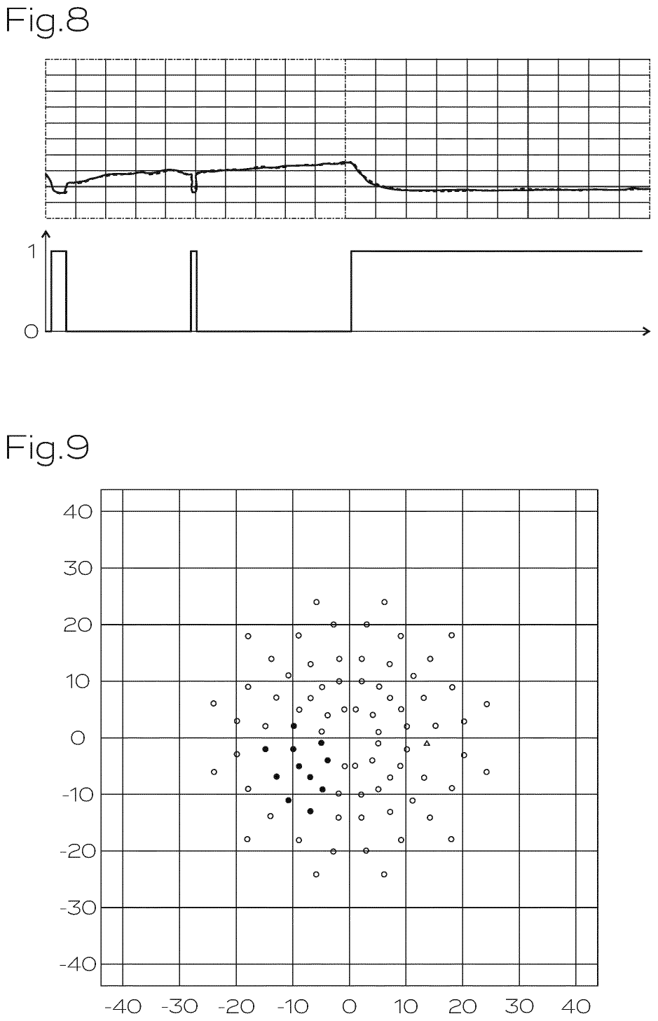

FIG. 8 shows a schematic view of a result of a pupillography with the examination device according to the invention;

FIG. 9 shows a schematic view of a result of a test of the visual field with the examination device according to the invention;

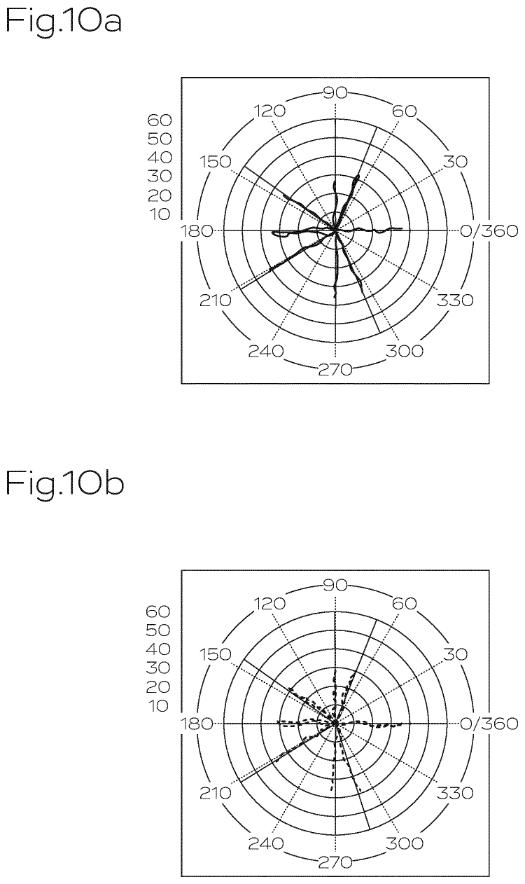

and FIGS. 10a and 10b show schematic diagrams of a result of a test of the eye movements with the examination device according to the invention.

FIG. 1a shows a schematic view of the head 7 of a person wearing the examination device 1. FIGS. 1b and 1c show schematic sectional views of FIG. 1a.

According to this embodiment, the examination device 1 comprises two spatially separated display devices 2, 2′, two sensing devices 5, 5′ and a detection device 6. Optical lenses are arranged between the display devices 2, 2′ and each eye, the optical lenses being designed such that the respective eye can focus on the images displayed by the display devices 2, 2′. For clarity, the lenses are not shown in the figures.

The display devices 2, 2′ are designed as screens of a visual output device (head-mounted display) to be worn on the head 7. It is fixed to the person and is a closed system so that the examination device 1 covers the entire visual range of the person. By fixing the examination device 1 to the head 7 of the person, the examination device 1 and the display devices 2, 2′ follow the head movements. According to this embodiment, the display devices 2, 2′ are designed as screens close to the eyes, with one screen in each case being visible exclusively for one eye 4, 4′. Thus, the examination device 1 and in particular the display devices 2, 2′ cover the entire field of view 8, 8′ of the person; the person is isolated from other visual impressions of the environment by the examination device 1.

Both sensing devices 5, 5′ are configured for gaze detection or oculography. According to this embodiment, the sensing devices 5, 5′ are designed as infrared camera with associated infrared light source (LEDs). The sensing devices 5, 5′ are each located in the portion of the examination device 1 associated with an eye 4, 4′ in order to be able to precisely sense the eye movements and the eye positions of the eyes 4, 4′.

The examination device 1 further comprises a detection device 6, which is configured to detect the movement of the head 7 of the person. According to this embodiment, the detection device 6 is designed as a gyrometer; in other embodiments of the invention, the detection device 6 may be designed as acceleration sensor or camera, in particular as inside-out camera or outside-in camera. Further, the examination device 1 is connected to an input device 9, which is adapted to receive manual inputs of the person in relation to the displayed images. The input device 9 may be a pushbutton which is connected to the examination device 1 via radio or via a wired interface.

To detect the pupil size 10 and the interpupillary distance 12 of the eyes 4, 4′, the detection device 6 described above is used, which is arranged inside the examination device 1.

Both the sensing devices 5, 5′ as well as the optical sensors for sensing the pupil size 10 and the interpupillary distance 12 and the detection device 6 are arranged inside or on the examination device 1 in such a way that the fields of view 8, 8′ associated with the two eyes 4, 4′ of the person are not disturbed. The examination device 1 is configured to transmit the received data, in particular the eye movement, the eye position, the pupil size 10, the interpupillary distance 12, and/or manual inputs of the person to an electronic data processing unit 3. The electronic data processing unit 3 is a conventional computer; the transmission is done via a wireless or wired interface, such as WLAN or USB.

FIG. 2 shows a schematic graphic view of a second embodiment of the examination device 1 according to the invention. The features of the embodiment according to FIG. 2 may preferably correspond to the features of the embodiment according to FIGS. 1a, 1b and/or 1c. According to this embodiment, two display devices 2, 2′ are provided, the display devices 2, 2′ being designed as projectors. The display devices 2, 2′ are arranged on the examination device 1 and/or are designed such that the images displayed by one display device 2, 2′ are only visible for one eye 4, 4′ of the person and the images displayed by the other display device 2, 2′ are only visible for the other eye 4, 4′ of the person.

FIG. 3 shows a schematic graphic view of a third embodiment of the examination device 1 according to the invention. The features of the embodiment according to FIG. 3 may preferably correspond to the features of the embodiment according to FIGS. 1a, 1b, 1c and/or 2. According to this embodiment, two display devices 2, 2′ are provided, the display devices 2, 2′ being designed as lasers. The lasers project the image into the eye 4, 4′ via at least one mirror surface each, the mirror surface being arranged in front of the eye 4, 4′ of the person. The mirror surfaces are designed such that they do not disturb the person's field of vision.

The display devices 2, 2′ are arranged on the examination device 1 and/or are designed such that the images displayed by one display device 2 are only visible for one eye 4 of the person and the images displayed by the other display device 2′ are only visible for the other eye 4′ of the person.

FIGS. 4a and 4b show schematic diagrams of a person's eye 4, 4′ and the respective pupil size 10. In FIG. 4a, the pupil size 10 is small in comparison to FIG. 4b. The pupil size 10 may be determined via the sensing device 5, 5′ or using a separate optical sensor.

FIGS. 5a and 5b show schematic diagrams of the interpupillary distance 12 of a person's eyes 4, 4′. In FIG. 5a, the eyes 4, 4′ of the person are focussed on an object that subjectively appears farther away compared to FIG. 5b.

FIG. 6 shows a schematic view of a result of a test of the saccades with the examination device 1 according to the invention. Saccades, also called “scanning jump in the course of gaze”, are rapid eye movements to a defined gaze target. In FIG. 6, the deflection of the eyes 4, 4′ is shown in degrees in the vertical direction over time in seconds. In other words, a positive angle indication means that the viewing direction of the eye 4, 4′ is directed to the right, and a negative angle indication means that the viewing direction of the eye 4, 4′ is directed to the left. The course of movement of the right eye 4, 4′ is shown in dashed lines and the course of movement of the left eye 4, 4′ is shown in dashed dots. The course of the light spot is shown as a solid line. It can be seen from the figures that the eyes 4, 4′ substantially follow the course of the light spot. To examine the saccades, the examination device 1 according to the invention can be used as follows: First, during a first measurement, a first light spot is displayed on the display device 2, 2′, in particular exclusively, the respective eye position of the eye 4, 4′ being sensed by the sensing device 5, 5′ when the eye 4, 4′ is focused on the first light spot.

It may be provided that the first light spot is displayed for a certain duration. In a subsequent further measurement, another light spot is displayed on the display device 2, 2′, in particular exclusively, which is arranged somewhere else compared to the first light spot, the respective eye position of the eye 4, 4′ being sensed with the sensing device 5, 5′ when the eye 4, 4′ is focused on the other light spot. It may be provided that the second light spot is displayed for a certain duration.

Optionally, the head movement, the pupil size 10, the interpupillary distance 12, and/or an eye movement may be sensed during and/or between the first and further measurements. The sensed eye positions, head movements, pupil sizes 10, interpupillary distance 12 and/or eye movements can be stored and/or evaluated by the data processing unit 3.

FIGS. 7a and 7b show schematic diagrams of a result of a strabismus diagnosis with the examination device 1 according to the invention. In both figures, the deflection of the left eye 4, 4′ in the horizontal direction is shown versus the deflection of the left eye 4, 4′ in the vertical direction. FIG. 7a shows the results of the examination for the left eye 4, 4′ with stimulation and FIG. 7b shows the results of the examination for the left eye 4, 4′ without stimulation. This means that the left eye 4, 4′ is entrained in a dissociated manner in this case.

According to this embodiment, without stimulation means that during the examination, images are displayed only to the right eye 4, 4′ via the display device 2, 2′ and the left eye is thus entrained in a dissociated manner.

In this case, with stimulation means that images are displayed to both the right eye 4, 4′ and the left eye 4, 4′ via the display device 2, 2′ during the examination. The present examination results are from the eye 4, 4′ to which an image was displayed. From the figures, it can be seen that the position of the eye with stimulation is different from the position of the eye without stimulation.

To examine the deviation with stimulation of both eyes 4, 4′, the examination device 1 according to the invention can be used as follows: First, a first image is displayed to both eyes 4, 4′ of the person by the display device 2, 2′, the image being designed in such a way that it appears to the person as one image. Here, the eye movements and in particular the eye positions of both eyes 4, 4′ are sensed by the sensing devices 5, 5′.

To examine the deviation with stimulation of only one eye 4, 4′, the examination device 1 according to the invention is used as follows: First, a first image is displayed to only one eye 4, 4′, in the present case only the right eye 4′, of the person by the display device 2, 2′. Here, the eye movements and in particular the eye positions of both eyes 4, 4′, in the present case only those of the left eye 4, are sensed by the sensing devices 5, 5′.

In both cases, the eye movements, the head movements, the interpupillary distance 12 and/or the pupil sizes 10 may be detected during the measurement, and the difference of the sensed eye positions may be calculated and/or output by the data processing unit 3. In the calculation, in both cases, the eye movements, the head movements, the interpupillary distance 12 and/or the pupil sizes 10 of the eyes 4, 4′ may be taken into account.

FIG. 8 shows a schematic view of a result of a pupillography with the examination device 1 according to the invention. In this figure, the first graph shows the variation of the pupil size 10 of the left 4 and the right eye 4′ over time. Pupillography is used to determine the function of the pupillary reflex, in particular the constriction during exposure to light, magnification in the dark and/or the aperture adjustment. The pupil size 10 of the right eye 4′ is shown as a dashed line and the pupil size 10 of the left eye 4 is shown as a solid line. In the graph below, the lumen value of the image is plotted versus time. The value 0 represents a low lumen value of the image, especially a black image.

The value 1 represents a high lumen value of the image in comparison, in particular for a white image. It can be seen from the figures that the pupil size 10 changes with respect to the lumen value of the displayed image.

To examine the pupillography of a person, the examination device 1 according to the invention can be used as follows: First, a first image is displayed to both eyes 4, 4′ of the person, the first image optionally being black or white, wherein a first pupil size 10 and/or a first interpupillary distance 12 of the eyes 4, 4′ are sensed with the sensing devices 5, 5′. It may be provided that the first image is displayed for a certain duration.

Subsequently, a further image is displayed to both eyes 4, 4′ of the person, the image being designed in such a way that it appears brighter or darker to the person than the first image, wherein a second pupil size 10 and/or a second interpupillary distance 12 of the eyes 4, 4′ are sensed with the sensing devices 5, 5′. It may be provided that the first image is displayed for a certain duration. During the measurement, the eye movements, the eye position, the interpupillary distance 12 and/or the head movements may optionally be detected. Furthermore, the difference of the sensed pupil sizes 10 and/or the sensed interpupillary distances 12 may be calculated by the data processing unit 3. Furthermore, the data processing unit 3 may optionally take into account the eye movements, the eye position, the interpupillary distance 12 and/or the head movements of the eyes 4, 4′ in this calculation.

FIG. 9 shows a schematic view of a result of a test of the visual field with the examination device 1 according to the invention. In this figure, the position of the stimulated retinal locus in the horizontal direction of the eye 4, 4′ is shown versus the position of the stimulated retinal locus in the vertical direction of the eye 4, 4′. The blind spot of the right eye 4′ is shown as triangle. Depending on the threshold value, the measuring points are displayed differently. Black points represent a high threshold value, i.e., these points had to be displayed brighter, in particular very bright and/or big, for the person to see them. White points represent a low threshold value.

It can be seen from the figures that the examined right eye 4′ of the person has a more significant sensitivity drop in one area of the visual field than in other areas of the visual field.

To examine the visual field of a person, the examination device 1 according to the invention can be used as follows: In a first measurement by the sensing device 5, 5′, a first eye position of the eye 4, 4′ is sensed, wherein a first light spot is displayed on the display device 2, 2′ in dependence on the sensed first eye position. In a further measurement, a second eye position of the eye 4, 4′ is sensed by the sensing device 5, 5′, wherein a different light spot is displayed on the display device 2, 2′ in dependence on the sensed second eye position. Furthermore, the manual inputs of the person are sensed with the input device 9 in relation to the displayed light spots.

During and/or between the first and the further measurements, the head movement, the eye movement, the interpupillary distance 12 and/or the pupil size 10 may optionally be detected. The manual inputs of the person in relation to the displayed light spots may be stored and/or evaluated by the data processing unit 3 taking into account the eye movement, the eye position, the interpupillary distance 12, the head movement and/or the pupil size 10.

FIGS. 10a and 10b show schematic diagrams of a result of a test of eye movement along eye muscle-specific axes with the examination device 1 according to the invention. In FIG. 10a, the deflection of the left eye 4, 4′ with stimulation in the horizontal direction is shown versus the deflection of the left eye 4, 4′ in the vertical direction. In FIG. 10b, the deflection of the left eye 4, 4′ without stimulation in the horizontal direction is shown versus the deflection of the left eye 4, 4′ in the vertical direction. Here, the left eye 4 is only entrained in a dissociated manner. Furthermore, the eye muscle axes are drawn as solid lines in FIGS. 10a and 10b. From these figures, the maximum deflections on the eye muscle-specific axes can be read.

From the figures, the maximum eye positions of the person's eyes 4, 4′ in the measured directions are shown. To examine the eye movement, in particular the eye mobility along eye muscle-specific axes, of a person, the examination device 1 according to the invention can be used as follows:

In a first measurement, a first light spot is displayed on the display device 2, 2′, in particular exclusively, wherein the eye position of the eye 4, 4′ is sensed by the sensing device 5, 5′ when the eye 4, 4′ focuses on the first light spot. Subsequently, in a further measurement, another light spot is displayed on the display device 2, 2′, in particular exclusively, which is closer to an edge of the respective display device 2, 2′ compared to the first light spot, wherein the eye position of the eye 4, 4′ is sensed with the sensing device 5, 5′ when the eye 4, 4′ focuses on the other light spot. These measurements are repeated until the, in particular maximum, eye movement could be determined with respect to the respective deflection direction.

Optionally, the head movement, the pupil size 10, the interpupillary distance 12 and/or the eye movement may be detected during and/or between the first and the further measurements. The sensed eye positions, head movements, pupil sizes 10, interpupillary distances 12 and/or eye movements may be stored and/or evaluated by the data processing unit 3.

Optionally, if substantially no difference is detected between the sensed eye positions, a maximum eye movement may be output and/or stored by the data processing unit 3.

### LIST OF REFERENCE SIGNS

1 examination device

2, 2′ display device

3 data processing unit

4, 4′ eye

5, 5′ sensing device

6 detection device

7 head

8, 8′ field of vision

9 input device

10 pupil size

11 pupil

12 interpupillary distance

1. An examination device for examining at least one eye of a person, the examination device comprising:
at least one optical sensing device configured to sense eye movements and eye positions of at least one eye of a person;
at least one optical display device designed to display or project images;
wherein the examination device is designed for the connection to an electronic data processing unit for storage and automated evaluation of the eye movements and eye positions in relation to the displayed images, or comprises such an electronic data processing unit; and
wherein the examination device is adapted to display an image by the display device prior to sensing the eye movements and eye positions by the sensing device, or prior to displaying an image by the display device, in order to bring at least one eye into a predefined eye position.

2. The examination device according to claim 1, wherein the examination device comprises at least one input device configured to sense manual inputs of the person in relation to the displayed images, wherein the data processing unit is adapted to store and evaluate the sensed inputs in relation to the displayed images.

3. The examination device according to claim 1, wherein the examination device comprises a detection device configured to detect head movements of the person, the data processing unit being adapted to take into account the sensed head movements in the evaluation of the eye movements, the eye positions and/or the inputs.

4. The examination device according to claim 1,
wherein the sensing device or a separate optical sensor is configured to sense the pupil size of the eye of the person, the data processing unit being adapted to take into account the sensed pupil size in the evaluation of the eye movements, the eye positions and/or the inputs,
and/or the sensing device or a separate optical sensor is configured to sense the interpupillary distance, wherein the data processing unit is adapted to take into account the sensed interpupillary distance in the evaluation of the eye movements, the eye positions and/or the inputs.

5. The examination device according to claim 1,
wherein the examination device comprises at least two spatially separated display devices, wherein the images displayed on one display device are only visible for one eye of the person and the images displayed on the other display device are only visible for the other eye of the person,
and/or wherein the examination device comprises at least two spatially separated display areas, wherein a first display area is only visible for one eye of the person and a second display area is only visible for the other eye of the person.

6. The examination device according to claim 1, wherein the examination device is adapted to be arranged on the head of a person and comprises a portable visual output device comprising two separately controllable display devices.

7. A method for automated eye examination with an examination device comprising at least one optical sensing device configured to sense eye movements and eye positions of at least one eye of a person and at least one optical display device designed to display or project images, the method comprising the steps of:
displaying an image by the display device and sensing an eye movement and an eye position of the eye by the sensing device,
storing and evaluating the at least one sensed eye movement and eye position and the at least one displayed image by an electronic data processing unit connected to the examination device, wherein,
prior to sensing the eye movement and eye position by the sensing device, an image is displayed by the display device to bring at least one eye into a predefined eye position, or
prior to displaying an image by the display device, an image is displayed by the display device to bring at least one eye into a predefined eye position.

8. The method according to claim 7, wherein the method comprises the following steps:
sensing an eye movement and an eye position of the eye by the sensing device,
displaying an image in dependence on the sensed eye movement and eye position of the eye by the display device, and
storing and evaluating the at least one sensed eye movement and eye position and the at least one displayed image by an electronic data processing unit connected to the examination device.

9. The method according to claim 7, wherein the method comprises the following steps:
displaying an image by the display device,
sensing an eye movement and an eye position of the eye in dependence on the displayed image by the sensing device, and
storing and evaluating the at least one sensed eye movement and eye position in relation to the at least one displayed image by an electronic data processing unit connected to the examination device.

10. The method according to claim 7,
wherein the displayed images comprise a first image component on which the eye is to focus, and a second image component, wherein
the displayed images comprise the same first image component and a different second image component each, and wherein
the image components are designed as light spots, which are different regarding their size, intensity, brightness and/or display duration.

11. The method according to claim 7, wherein the method comprises the following further steps:
recording manual inputs of the person in relation to the displayed images by an input device connected to the examination device, and
storing and evaluating the recorded inputs in relation to the displayed images by the data processing unit.

12. The method according to claim 7, wherein the method comprises the following further steps:
recording head movements of the person by a detection device connected to the examination device, and
taking into account the sensed head movements by the data processing unit in the evaluation of the eye movements, eye positions and/or inputs.

13. The method according to claim 7, wherein the examination device is arranged on the head of the person so that the examination device and the display device follow the head movements of the person.

14. The method according to claim 7,
wherein the eye movements and the eye positions of both eyes of the person are sensed substantially simultaneously using one or more sensing device/s,
and/or wherein images are displayed substantially simultaneously for both eyes of the person.

15. The method according to claim 7,
wherein the pupil size of the eye is determined by the sensing device or by a separate optical sensor, wherein the eye movements, the eye positions, the sensed pupil size and/or the inputs are taken into account by the data processing unit in the evaluation,
and/or the interpupillary distance is determined by the sensing device or by a separate optical sensor, wherein the eye movements, the eye positions, the sensed interpupillary distance and/or the inputs are taken into account by the data processing unit in the evaluation.
